(19) United States
(12) Patent Application Publication
(10) Pub. No.: US 2023/0108128 A1
(43) Pub. Date: Apr. 6, 2023

(54) PREDETERMINED CALIBRATION CURVE

(71) Applicant: PROCISEDX INC, San Diego, CA (US)

(72) Inventor: MICHAEL HALE, San Diego, CA (US)

(73) Assignee: PROCISEDX INC, San Diego, CA (US)

(21) Appl. No.: 17/992,828

(22) Filed: Nov. 22, 2022

Related U.S. Application Data
(63) Continuation of application No. PCT/US2021/033991, filed on May 25, 2021.
(60) Provisional application No. 63/031,168, filed on May 28, 2020.

(51) Int. Cl. G01N 33/96; G01N 21/64; G01N 33/542

(52) U.S. Cl. CPC G01N 33/96; G01N 21/6408; G01N 21/6428; G01N 33/542; G01N 2201/12746; G01N 2496/00

(57) ABSTRACT

Methods and compositions are disclosed for preparing a predetermined standardized calibration curve, which can be stored on a label, barcode or tag. A lyophilized product containing assay reagents for use in a bioassay and a known amount of analyte. A predetermined calibration curve associated with the lyophilized product. A predetermined calibration curve makes the preparation of a new calibration curve unnecessary. The methods and compositions are useful in bioassays such as a FRET assay, a real-time reverse transcription polymerase (RT-PCR) assay, a chemiluminescence assay, or any other bioassay that elicits a detectable response based on a change in, or appearance of, color, fluorescence, or reflectance.

### CROSS-REFERENCE TO RELATED APPLICATION

The current application is a continuation application of PCT/US2021/033991, filed May 25, 2021, which application claims priority to U.S. Provisional Pat. Application No. 63/031,168, filed May 28, 2020, the contents both of which are hereby incorporated by reference in their entities for all purposes.

### BACKGROUND

For analytical instruments, a calibration curve is a linear relationship between the concentration of an analyte, which is an independent variable, with the instrument’s response, which is a dependent variable. The instrument’s response can be an optical response. This linear relationship is useful for determining unknown concentrations of an analyte in a sample. A concentration determination exercise can use a calibration curve with response values for different concentrations of an analyte. By determining the relationship between the magnitude of an optical signal (response value) for a known amount of analyte in a standard for several samples, the linear relationship (the calibration curve) is used to estimate the amount of that specific analyte in a sample of unknown concentration.

Typically, a calibration curve is instrument specific and therefore, a calibration curve can be generated before measuring unknown samples or test samples on the instrument. So even though the instrument is the same model and type, a new calibration curve is generated when the instrument is used in a daily routine.

If however, one instrument or device can be used for each instrument in the field, there would be a significant decrease in time required to obtain a calibration curve and a significant reduction in time and resources. A predetermined calibration curve that is applicable to a wide variety of instruments would be a considerable cost and resource saver. There is a need in the art for a predetermined calibration curve that allows an end user’s system to be adjusted to give the same calibrated output as that of the system used to derive the predetermined calibration curve. In addition, lesser volumes of calibrator and/or control compositions would be required. The current disclosure satisfies these and other needs and offers other advantages as well.

### BRIEF SUMMARY

In one embodiment, the present disclosure provides a method for preparing and storing a predetermined standardized curve for an assay, the method comprising:
(a) preparing a plurality of lyophilized calibrator compositions in an assay device, wherein the assay device has a plurality of wells or is a single cuvette;
(b) incubating the assay device in an analyzer;
(c) obtaining an optical signal for each of the plurality of standard calibrator compositions to generate a plurality of optical signals;
(d) preparing a standardized curve of the analyte from the plurality of optical signals; and
(e) storing or assigning the predetermined standardized curve onto a label, barcode or tag for the assay device.

Advantageously, the predetermined standardized curve can be loaded onto a label, a barcode, or tag (such as RFID tag). The barcode can be read by a barcode reader to obtain the calibration curve.

In another embodiment, the present disclosure provides a method for adjusting a predetermined standardized curve of an analyte for an assay, the method comprising:
(a) measuring a signal for a calibrator composition, wherein the calibrator composition comprises a known amount of the analyte within the predetermined standardized curve;
(b) obtaining a ratio of the signal for the calibrator composition; and
(c) adjusting the predetermined standardized curve according to the ratio obtained.

In certain instances, the method further comprises (d) optionally determining an unknown analyte concentration according to the adjusted predetermined standardized curve.

In certain instances, the ratio of the signal for the calibrator composition is determined by dividing the signal output from the analyzer by the predetermined output from the standardized curve. In other instances, the ratio is determined by dividing the predetermined output from the standard curve by the signal output from the analyzer. The predetermined output can be loaded onto a label, a barcode, or tag (such as RFID tag). The barcode can be read by a barcode reader to obtain the predetermined output.

In certain instances, the calibrator composition is a plurality of calibrator compositions.

In certain instances, the ratio is used to normalize other analyzers such as the same type of analyzer in a different location.

In another embodiment, the present disclosure provides a lyophilized product, the lyophilized product comprising:
a fluorophore acceptor attached to a first antibody having a first epitope for an analyte;
a fluorophore donor attached to a second antibody having a second epitope for the analyte; and a known amount of analyte.

In certain aspects, the lyophilized product is a bead or a plurality of beads, wherein each bead has a different concentration of analyte. In certain aspects, the lyophilized product is an array of beads (two or more).

In certain aspects, the analyte is a member selected from the group consisting of a protein, a nucleic acid, an autoantibody and a vitamin.

In certain aspects, the lyophilized product is reconstituted with a diluent.

In certain aspects, the lyophilized product is reconstituted with a diluent in a dilution series.

In certain aspects, the dilution series is used to generate a standard curve.

In certain aspects, the lyophilized product is disposed in an assay device.

In certain aspects, the assay device is a member selected from the group of a 96, 384 and 1536 well plate or a cuvette.

In another embodiment, the present disclosure provides a method for verifying the accuracy of a predetermined standardized curve for an assay, the method comprising:
(a) reconstituting a lyophilized product in an assay device to form a calibrator composition wherein the lyophilized product contains a fluorophore acceptor attached to a first antibody having a first epitope for an analyte; a fluorophore donor attached to a second antibody having a second epitope for the analyte; and a known amount of analyte within the predetermined standardized curve, wherein the assay device has a plurality of wells;
(b) incubating the assay device in an analyzer;
(c) obtaining an optical signal for the calibrator composition; and
(d) comparing the known amount of analyte to the predetermined standardized curve.

In certain aspects, the lyophilized product is a bead.

In certain aspects, the bead is a plurality of beads, wherein each bead has a different concentration of analyte.

In certain aspects, the analyte is a member selected from the group consisting of a protein, a nucleic acid, an autoantibody and a vitamin.

In certain aspects, the assay device is a member selected from the group of a 96, 384 and 1536 well plate or a cuvette.

In certain aspects, the lyophilized product is used to make a dilution series of the analyte.

In another embodiment, the present disclosure provides method for making a standardized curve for an assay, the method comprising:
(a) preparing a plurality of calibrator compositions in an assay device each calibrator composition containing a fluorophore acceptor attached to a first antibody having a first epitope for an analyte; a fluorophore donor attached to a second antibody having a second epitope for the analyte, and a known amount of analyte, wherein the assay device has a plurality of wells;
(b) incubating the assay device in an analyzer;
(c) obtaining an optical signal for each of the plurality of standard calibrator compositions to form a plurality of optical signals; and
(d) preparing a standardized curve of the analyte from the plurality of optical signals.

In certain aspects, each of the plurality calibrator compositions is contained within a single well of the assay device.

In certain aspects, the analyte is a member selected from the group consisting of a protein, a nucleic acid, a vitamin and an autoantibody.

These and other embodiments, aspects and advantages will become more apparent when read with the detailed description and figures, which follow.

### BRIEF DESCRIPTION OF THE DRAWINGS

FIG. 1 is an assay device of the present disclosure.

FIG. 2 is an illustration of an assay.

FIG. 3 is a predetermined standard curve of the present disclosure.

FIG. 4A is an illustration of an embodiment of the disclosure.

FIG. 4B is an illustration of an embodiment of the disclosure.

### DETAILED DESCRIPTION

### I. Definitions

The terms “a,” “an,” or “the” as used herein not only includes aspects with one member, but also includes aspects with more than one member.

The term “about” as used herein to modify a numerical value indicates a defined range around that value. If “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

When the modifier “about” is applied to describe the beginning of a numerical range, it applies to both ends of the range. Thus, “from about 500 to 850 nm” is equivalent to “from about 500 nm to about 850 nm.” When “about” is applied to describe the first value of a set of values, it applies to all values in that set. Thus, “about 580, 700, or 850 nm” is equivalent to “about 580 nm, about 700 nm, or about 850 nm.”

“Fluorescence resonance energy transfer (FRET)” or “Forster resonance energy transfer (FRET)” refer to a mechanism describing energy transfer between a donor compound such as cryptate and an acceptor compound such as Alexa 647, when they are in proximity to one another and when they are excited at the excitation wavelength of the donor fluorescent compound. A donor compound, initially in its electronic excited state, may transfer energy to an acceptor fluorophore through nonradiative dipole-dipole coupling. The efficiency of this energy transfer is inversely proportional to the sixth power of the distance between donor and acceptor, making FRET extremely sensitive to small changes in distance.

“FRET partners” refers to a pair of fluorophores consisting of a donor fluorescent compound such as cryptate and an acceptor compound such as Alexa 647, when they are in proximity to one another and when they are excited at the excitation wavelength of the donor fluorescent compound, these compounds emit a FRET signal. It is known that, in order for two fluorescent compounds to be FRET partners, the emission spectrum of the donor fluorescent compound must partially overlap the excitation spectrum of the acceptor compound. The preferred FRET-partner pairs are those for which the value R0 (Förster distance, distance at which energy transfer is 50% efficient) is greater than or equal to 30 Å.

“FRET signal” refers to any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent.

As used herein, “standard samples” are samples containing an analyte standard at a specific concentration.

A “calibration standard” includes a known or predetermined amount of an analyte standard in combination with a known constant amount of one or more reagents. These allow an end user’s system to be adjusted to give the same calibrated output as that of the system used to derive the predetermined calibration curve.

As used herein, “lyophilized product” is a composition or product where water has been removed by a process typically referred to as freeze drying. Lyophilization works by freezing the product, then reducing the pressure and adding heat to allow the frozen water in the product to sublimate.

As used herein, the term “calver” is a product (e.g. a lyophilized product) that can be used as both a calibrator control and a verifier control in an assay and is a so called “calver.”

An “analyte” as used herein can include a natural or synthetic molecule for use in biological systems. Suitable analytes include a protein, a peptide, an enzyme substrate, a hormone, an antibody or a fragment thereof, an autoantibody, an antigen, a hapten, an avidin, a streptavidin, a carbohydrate, a carbohydrate derivative, an oligosaccharide, a polysaccharide, a nucleic acid, a deoxynucleic acid, a fragment of DNA, a fragment of RNA, vitamin and the like.

A “detectable response” or “optical signal” as used herein includes a change in, or occurrence of, a parameter in a bioassay or a test system that is capable of being perceived, either by direct observation or instrumentally, and which is a function of the presence of a specifically targeted member or analyte in a sample. Such detectable responses include a change in, or appearance of, color, fluorescence, reflectance, pH, chemiluminescence, infrared spectra, magnetic properties, radioactivity, light scattering, or x-ray scattering.

### II. Embodiments

In certain aspects, a predetermined standardized calibration curve advantageously reduces the number of calibration standards that are required to be prepared by the end user. A predetermined calibration curve makes the preparation of a new calibration curve unnecessary. The methods and compositions described herein are useful in bioassays such as a FRET assay, a real-time reverse transcription polymerase (RT-PCR) assay, a chemiluminescence assay, or any other bioassay that elicits a detectable response based on a change in, or appearance of, color, fluorescence, or reflectance.

In one embodiment, the disclosure provides a method for preparing and storing a standardized curve for an assay, the method comprising:
(a) preparing a plurality of lyophilized calibrator compositions in an assay device, wherein the assay device has a plurality of wells or a single cuvette;
(b) incubating the assay device in an analyzer;
(c) obtaining an optical signal for each of the plurality of standard calibrator compositions to form a plurality of optical signals;
(d) preparing a standardized curve of the analyte from the plurality of optical signals; and
(e) storing or assigning the predetermined standardized curve onto a label, a barcode or tag on the assay device.

The assay device can be a single well (cuvette), a 96 well plate, a 384 well plate, or 1536-well plate.

In certain instances, each calibrator composition contains a first antibody having a first epitope for an analyte; a second antibody having a second epitope for the analyte, and a known amount of analyte (antigen). A fluorophore acceptor can be attached to the first antibody and a fluorophore donor can be attached to the second antibody.

Alternatively, in certain instances, each calibrator composition contains an antibody having an acceptor fluorophore for the analyte and a known amount of analyte (antigen) having a donor fluorophore. In certain instances, the antibody has a donor fluorophore and the analyte (antigen) has an acceptor fluorophore.

A plurality of optical signals is obtained and a standardized curve of the analyte from the plurality of optical signals is generated. The predetermined standardized curve can be loaded onto a barcode or RFID tag or label. The barcode can be read by a barcode reader.

For a typical analytical assay, a predetermined standard curve for the assay is generated. This generally includes a series of samples with known concentration amounts of analytes and known amounts of each reagent. For example, a calibration curve for fecal calprotectin can be obtained by using the following procedure. Six calibrator compositions having, respectively, 0.00, 15.62, 31.25, 62.50, 125.00, and 250.00 µg/mL of calprotectin can be used. The test sample is placed in an analyzer and an optical readout is obtained.

In certain instances, the calibrators can be for example, the calibrator compositions having, respectively, 0.00, 15.62, 31.25, 62.50, 125.00, or 250.00 µg/mL of calprotectin previously discussed.

In certain aspects, a predetermined calibration assay is associated with a lyophilized product. The lyophilized product can be a bead or particle and disposed within an assay device. As an example, the assay device contains a lyophilized product (a calver) comprising 15.62 µg/mL of calprotectin along with the specific reagent amounts to measure this amount of analyte. The end user measures the calver and obtains an optical readout. The optical readout corresponds to 15.62 ± 0.10 µg/mL of calprotectin. Small adjustments can be made to the analyte concentration amounts to ensure the optical readout of the user’s instrument is correlated to 15.62 µg/mL of the predetermined calibration curve. If the instrument’s readout or optical response corresponds to 15.62 µg/mL of calprotectin, no adjustment to the instrument is necessary.

In another embodiment, the present disclosure provides a method for adjusting a predetermined standardized curve of an analyte for an assay, the method comprising:
(a) measuring a signal for a calibrator composition, the calibrator composition comprising a known amount of the analyte within the predetermined standardized curve;
(b) obtaining a ratio of the signal for the calibrator composition; and
(c) adjusting the predetermined standardized curve according to the ratio obtained.

In certain instances, the method further comprises (d) optionally determining an unknown analyte concentration according to the adjusted predetermined standardized curve.

In certain instances, the calibrator composition is a plurality of calibrator compositions.

In certain instances, the ratio is determined by dividing the actual signal output from the analyzer by the predetermined output from the standard curve. In other instances, the ratio is determined by dividing the predetermined output by the actual analyzer output.

In certain instances, the ratio is used to normalize analyzers in different locations.

In certain other embodiments, the present disclosure provides a product disposed in an assay device. The product can be a lyophilized product. Suitable assay devices include for example, a cuvette or an array of wells, such as a multi-well plate. Various multi-well plates include a multi-well of 2 to 2000 wells such as 96-well plate, a 384-well plate, a 1536 well-plate, a single cuvette, or the like. A predetermined calibration curve is associated with the lyophilized product disposed in the assay device. A barcode, a label, or tag having the predetermined calibration curve encoded therein can be attached to assay device.

In certain instances, the product disposed in the assay device contains various reagents. The reagents are useful for an analytical assay such as a bioassay performed on an instrument. The product can also contain or comprise an analyte, which may be the analyte determined in or by the bioassay. The reagents are specific for the bioassay and a predetermined calibration curve. By including a product in the assay device along with specific reagents, a predetermined calibration curve can be used in lieu of the user developing a calibration curve de novo. In certain instances, a lyophilized product having known concentrations of analyte and assay components can be reconstituted and subsequently used with a pre-determined calibration curve.

In one embodiment, the present disclosure provide a lyophilized product, the lyophilized product containing:
a fluorophore acceptor attached to a first antibody having a first epitope for an analyte;
a fluorophore donor attached to a second antibody having a second epitope for the analyte; and a known amount of analyte.

In another embodiment, the present disclosure provide a lyophilized product, the lyophilized product containing: an antibody having an acceptor fluorophore for an analyte and a known amount of analyte (antigen) having a donor fluorophore. In certain instances, the antibody has a donor fluorophore and the analyte (antigen) has an acceptor fluorophore.

In certain aspects, the lyophilized product is a bead or a pellet. In certain aspects, the bead is a plurality of beads or an array of beads, wherein each bead has a different concentration of analyte. Suitable analytes include for example, a biomolecule, an antibody, an autoantibody, an enzyme, a carbohydrate, a nucleic acid, a protein, or a vitamin. A lyophilized product can be a calibration standard.

In one aspect, a lyophilized product is disposed in an assay device such as by lyophilizing a uniform and specific amount of reagents including an analyte of the proposed assay (e.g., donor and acceptor conjugate) into one or more wells of a multi-well plate. A predetermined calibration curve is previously established for the product deposited in the plate. The calibration curve is passed along with the plate such as with a bar code and is specific for that analyte contained within the product.

In certain instances, the analyte is an anti-TNFα drug with a reconstituted drug concentration level of about 1.0 to about 100 ng/10 µL.

In certain instances, the analyte is human serum albumin with a concentration of about 3 g/L to about 500 g/L.

In certain instances, the analyte is vitamin D with a concentration level of about 2 ng/mL to about 500 ng/mL.

In certain instances, the analyte is C-reactive protein with a concentration of about 3 mg/L to about 200 mg/L or higher.

In certain aspects, the analyte is VCAM-1 with a concentration of about 100 ng/mL to about 500 ng/mL or higher such as 1500 ng/mL.

In certain aspects, the analyte is α 2-MG, which is a plasma protein with a concentration of about 0.1 mg/mL to about 10 mg/mL.

In certain aspects, the analyte is calprotectin with a concentration range of about 10 µg/g to about 800 µg/g.

When in use, for a particular assay, a set number (1 or more) of calver(s) will be run to determine whether the predetermined calibration curve is used without change, or whether the calibration curve is adjusted or off-set based on the values of the calver(s). A calver is used as both a calibration control and a verification control of the assay.

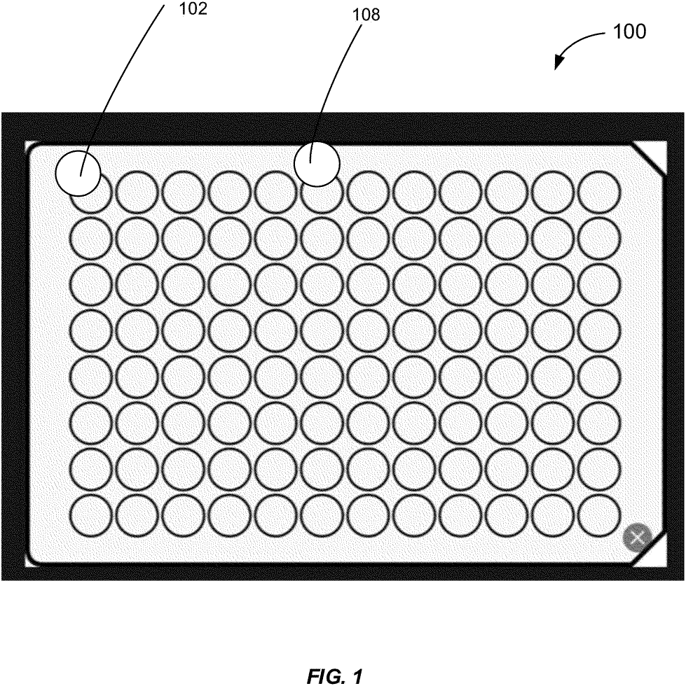

FIG. 1 illustrates is a 96-well plate 100 having a lyophilized product 102 in one or more wells. The product 102 can be in the form of a bead or pellet or a generally spherical shape. The lyophilized product typically includes reagents and an analyte of a diagnostic assay to be performed using the assay device. The lyophilized product can include additional components such as one or more binding agents, salts, or buffers. In certain instances, the lyophilzed product comprises an known amount of analyte, a fluorophore acceptor attached to a first antibody having a first epitope for the analyte; and a fluorophore donor attached to a second antibody having a second epitope for the analyte.

In certain aspects, the assay device 100 can include more than one lyophilized product within another of the wells. The second lyophilized product 108 can also be in the form of a bead or pellet, or a spherical shape. The second lyophilized product can be identical to the first lyophilized product. The second lyophilized product can also include more than one reagent of the diagnostic assay. In some embodiments, the second lyophilized product is a bead or pellet formed by lyophilization of a solution that includes reagents and an analyte.

The reagents can be any component of an assay of interest. The lyophilized products can include a known amount of reagents. The reagents can be, for example, enzymes, inorganic catalysts, dyes, binding agents, tags, antibodies, nucleic acid primers, probes or other nucleic acid constructs, cofactors, or ligands. In some aspects, the reagents include one or more labels. In this context, the term “label” refers to compositions detectable by spectroscopic, photochemical, biochemical, immunochemical, chemical, or other physical means. Useful labels include fluorescent dyes (fluorophores), fluorescent quenchers, luminescent agents, electron-dense reagents, enzymes (e.g., as commonly used in an ELISA), biotin, digoxigenin, ³²P and other isotopes, haptens, proteins, nucleic acids, or other substances which may be made detectable, e.g., by incorporating a label into or linking a label to an oligonucleotide, peptide, or antibody specifically reactive with a target molecule. The terms include combinations of single labeling agents, e.g., a combination of fluorophores that provides a unique detectable signature, e.g., at a particular wavelength or combination of wavelengths. In some embodiments, one or more of the reagents include one or more chromophores. In some embodiments, one or more of the reagents include one or more fluorophores. In some embodiments, one or more of the reagents include one or more quenchers.

In some embodiments, one or more of the reagents include one or more cryptate dyes or conjugates of cryptate dyes. Cryptates are complexes that include a macrocycle within which a lanthanide ion such as terbium or europium is tightly embedded or chelated. This cage like structure is useful for collecting irradiated energy and transferring the collected energy to the lanthanide ion. The lanthanide ion can release the energy with a characteristic fluorescence. U.S. Pat. Nos. 6,406,297, 6,515,113, 6,864,103, 8,507,199 and 8,173,800, as well as International Patent Application No. WO 2015/157057 disclose cryptate dyes and are hereby incorporated by reference.

Cryptates can be used in various diagnostic assays. Some assays rely on time-resolved fluorescence resonance energy transfer (TR-FRET) mechanisms where two fluorophores are used. In these assays, energy is transferred between a donor fluorophore and an acceptor fluorophore if the two fluorophore are in close proximity to the each other. Excitation of the donor (cryptate) by an energy source (e.g., UV light) produces an energy transfer to the acceptor if the two fluorophores are within a given proximity. In turn, the acceptor emits light at its characteristic wavelength. In order for TR-FRET to occur, the fluorescence emission spectrum of the donor molecule must overlap with the absorption or excitation spectrum of the acceptor chromophore. Moreover, the fluorescence lifetime of the donor molecule must be of sufficient duration to allow the TR-FRET to occur.

In certain aspects, the lyophilized product is reconstituted with a diluent. For example, the product can be reconstituted with a buffer or saline. The lyophilized product can be diluted and tested to ensure that a preprogramed or pre-determined standard curve is accurate and useful.

In other aspects, the diluent in a dilution series. A dilution series can be used to generate a standard curve or to ensure that a preprogramed or pre-determined standard curve is accurate and useful.

In certain aspects, the disclosure allows for less wells to be used during testing and accommodates for differences in plate reader outputs. Each well containing lyophilized material can be sealed individually allowing it to be opened just prior to use. A convenient seal is a plastic cover or transparent film covering the assay device or plate.

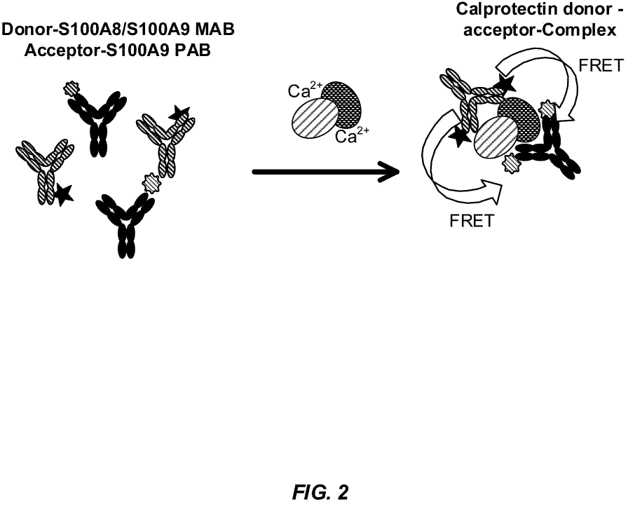

If one anti-calprotectin antibody is labeled with a donor fluorophore and a second anti-calprotectin antibody is labeled with an acceptor fluorophore, TR-FRET can occur in the presence of the calprotectin antigen (i.e., analyte, FIG. 2). The increase in FRET signal of the acceptor is proportional to the level of calprotectin present in the patient’s sample (e.g., stool or fecal sample) as interpolated from a known amount of calprotectin calibrators.

A predetermined calibration curve is associated with a lyophilized product such as with a label, a bar code or tag. In one embodiment, the present disclosure provide a method for making a predetermined standardized curve for an assay, the method comprising:
(a) preparing a plurality of calibrator compositions in an assay device each calibrator composition containing a fluorophore acceptor attached to a first antibody having a first epitope for an analyte; a fluorophore donor attached to a second antibody having a second epitope for the analyte, a known amount of analyte, wherein the assay device has a plurality of wells or a single cuvette;
(b) incubating the assay device in an analyzer;
(c) obtaining an optical signal for each of the plurality of standard calibrator compositions to form a plurality of optical signals; and
(d) preparing a predetermined standardized curve of the analyte from the plurality of optical signals.

Advantageously, the predetermined standardized curve is associated with an assay device containing a lyophilized product.

In another embodiment, the present disclosure provide a method for verifying the accuracy of a predetermined standardized curve for an assay, the method comprising:
(a) reconstituting a lyophilized product in an assay device to form a calibrator composition wherein the lyophilized product contains a fluorophore acceptor attached to a first antibody having a first epitope for an analyte; a fluorophore donor attached to a second antibody having a second epitope for the analyte; and a known amount of analyte within the predetermined standardized curve, wherein the assay device has a plurality of wells;
(b) incubating the assay device in an analyzer;
(c) obtaining an optical signal for the calibrator composition; and
(d) comparing the known amount of analyte to the predetermined standardized curve.

In certain aspects, the present disclosure provides a kit. In one aspect, the kit comprises a packaged combination of materials, typically intended for use in conjunction with each other. Kits in accordance with the disclosure can include instructions or other information in a “tangible” form such as printed information, electronically recorded on a computer-readable medium, or otherwise recorded on a machine-readable medium such as a barcode for storing numerical values.

In order to detect a FRET signal, a FRET acceptor is required. The FRET acceptor has an excitation wavelength that overlaps with an emission wavelength of the FRET donor. The FRET signal of the acceptor is proportional to the concentration level of analyte present in the sample, such as a patient’s blood sample as interpolated from a known amount of calibrators i.e., a standard curve. A cryptate donor can be used to label the first antibody AB-1. Lumi4 has 3 spectrally distinct peaks, at 490, 550 and 620 nm, which can be used for energy transfer (FIG. 9). An acceptor can be used to label the second antibody AB-2. The acceptor molecules that can be used include, but are not limited to, fluorescein-like (green zone) acceptor, Cy5, DY-647, Alexa Fluor 488, Alexa Fluor 546, Allophycocyanin (APC), and Phycoeruythrin (PE) and Alexa Fluor 647. Donor and acceptor fluorophores can be conjugated using a primary amine on an antibody.

In certain aspects of the embodiments, the assay uses a donor fluorophore consisting of terbium bound within a cryptate. The terbium cryptate can be excited with a 365 nm UV LED. The terbium cryptate emits at four (4) wavelengths within the visible region. In one aspect, the assay uses the lowest donor emission energy peak of 620 nm as the donor signal within the assay. In certain aspects, the acceptor fluorophore, when in very close proximity, is excited by the highest energy terbium cryptate emission peak of 490 nm causing light emission at 520 nm. Both the 620 nm and 520 nm emission wavelengths are measured independently in a device or instrument and results can be reported as RFU ratio 620/520.

### III. Device

Various instruments and devices are suitable for use in the present disclosure. Many spectrophotometers have the capability to measure fluorescence. Fluorescence is the molecular absorption of light energy at one wavelength and its nearly instantaneous re-emission at another, longer wavelength. Some molecules fluoresce naturally, and others must be modified to fluoresce.

A fluorescence spectrophotometer or fluorometer, fluorospectrometer, or fluorescence spectrometer measures the fluorescent light emitted from a sample at different wavelengths, after illumination with light source such as a xenon flash lamp. Fluorometers can have different channels for measuring differently-colored fluorescent signals (that differ in their wavelengths), such as green and blue, or ultraviolet and blue, channels. In one aspect, a suitable device includes an ability to perform a time-resolved fluorescence resonance energy transfer (FRET) experiment.

Suitable fluorometers can hold samples in different ways, including cuvettes, capillaries, Petri dishes, and microplates. The assays described herein can be performed on any of these types of instruments. In certain aspects, the device has an optional microplate reader, allowing emission scans in up to 384-well plates. Others models suitable for use hold the sample in place using surface tension.

Time-resolved fluorescence (TRF) measurement is similar to fluorescence intensity measurement. One difference, however, is the timing of the excitation / measurement process. When measuring fluorescence intensity, the excitation and emission processes are simultaneous: the light emitted by the sample is measured while excitation is taking place. Even though emission systems are very efficient at removing excitation light before it reaches the detector, the amount of excitation light compared to emission light is such that fluorescent intensity measurements exhibit elevated background signals. The present disclosure offers a solution to this issue. Time resolve FRET relies on the use of specific fluorescent molecules that have the property of emitting over long periods of time (measured in milliseconds) after excitation, when most standard fluorescent dyes (e.g., fluorescein) emit within a few nanoseconds of being excited. As a result, it is possible to excite cryptate lanthanides using a pulsed light source (e.g., Xenon flash lamp or pulsed laser), and measure after the excitation pulse.

As the donor and acceptor fluorescent compounds attached to the antibodies move closer together, an energy transfer is caused from the donor compound to the acceptor compound, resulting in a decrease in the fluorescence signal emitted by the donor compound and an increase in the signal emitted by the acceptor compound, and vice-versa. The majority of biological phenomena involving interactions between different partners will therefore be able to be studied by measuring the change in FRET between two fluorescent compounds coupled with compounds which will be at a greater or lesser distance, depending on the biological phenomenon in question.

The FRET signal can be measured in different ways: measurement of the fluorescence emitted by the donor alone, by the acceptor alone or by the donor and the acceptor, or measurement of the variation in the polarization of the light emitted in the medium by the acceptor as a result of FRET. One can also include measurement of FRET by observing the variation in the lifetime of the donor, which is facilitated by using a donor with a long fluorescence lifetime, such as rare earth complexes (especially on simple equipment like plate readers). Furthermore, the FRET signal can be measured at a precise instant or at regular intervals, making it possible to study its change over time and thereby to investigate the kinetics of the biological process studied.

In certain aspects, the device disclosed in PCT/IB2019/051213, filed Feb. 14, 2019 is used, which is hereby incorporated by reference. That disclosure in that application generally relates to analyzers that can be used in point-of-care settings to measure the absorbance and fluorescence of a sample with minimal or no user handling or interaction. The disclosed analyzers provide advantageous features of more rapid and reliable analyses of samples having properties that can be detected with each of these two approaches. For example, it can be beneficial to quantify both the fluorescence and absorbance of a blood sample being subjected to a diagnostic assay. In some analytical workflows, the hematocrit of a blood sample can be quantified with an absorbance assay, while the signal intensities measured in a FRET assay can provide information regarding other components of the blood sample.

One apparatus disclosed in PCT/IB2019/051213 is useful for detecting an emission light from a sample, and absorbance of a transillumination light by the sample, which comprises a first light source configured to emit an excitation light having an excitation wavelength. The apparatus further comprises a second light source configured to transilluminate the sample with the transillumination light. The apparatus further comprises a first light detector configured to detect the excitation light, and a second light detector configured to detect the emission light and the transillumination light. The apparatus further comprises a dichroic mirror configured to (1) epi-illuminate the sample by reflecting at least a portion of the excitation light, (2) transmit at least a portion of the excitation light to the first light detector, (3) transmit at least a portion of the emission light to the second light detector, and (4) transmit at least a portion of the transillumination light to the second light detector.

One suitable cuvette for use in the present disclosure is disclosed in PCT/IB2019/051215, filed Feb. 14, 2019. One of the provided cuvettes comprises a hollow body enclosing an inner chamber having an open chamber top. The cuvette further comprises a lower lid having an inner wall, an outer wall, an open lid top, and an open lid bottom. At least a portion of the lower lid is configured to fit inside the inner chamber proximate to the open chamber top. The lower lid comprises one or more (e.g., two or more) containers connected to the inner wall, wherein each of the containers has an open container top. In certain aspects, the lower lid comprises two or more such containers. The lower lid further comprises a securing means connected to the hollow body. The cuvette further comprises an upper lid wherein at least a portion of the upper lid is configured to fit inside the lower lid proximate to the open lid top.

### IV. EXAMPLES

### Example 1: Collection of Stools and Preparation of Stool Extracts

Stools are collected in plastic containers and immediately frozen below -20° C. In order to prepare extracts, the stools are thawed and 5 grams aliquots are collected, suspended with 10 ml of fecal extraction buffer and homogenized on ice for one minute at 20000 rpm, using an mechanical homogenizer. The temperature is maintained between 20° C. and 23° C. during this procedure. The homogenates are centrifuged at 45000 g for 20 minutes at 4° C. and the top halves of the supernatants are pipetted off and can be used.

This example illustrates a method of this disclosure detecting the presence and amount of calprotectin in a trFRET assay. As shown in FIG. 2, calprotectin binds to an anti-calprotection antibody (MAB-1) labeled with a donor fluorophore and a second antibody (MAB-2) labeled with acceptor fluorophore. The calprotectin analyte is in a sample from a patient (i.e., fecal sample, prepared as above) and it binds to both anti-calprotectin antibodies simultaneously resulting in a dual labeled calprotectin. After light excitation a FRET signal occurs and is detected.

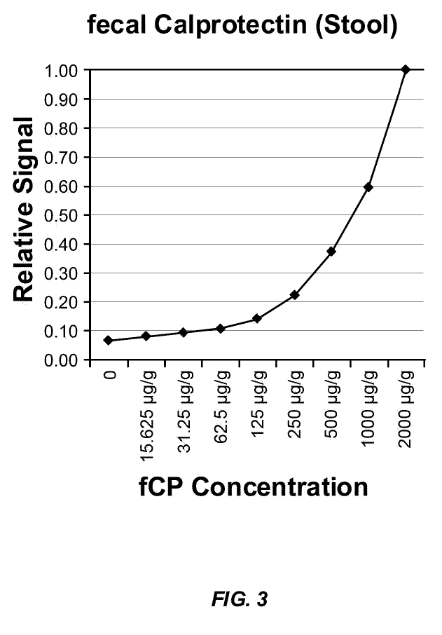

If one anti-calprotectin antibody is labeled with a donor fluorophore and a second anti-calprotectin antibody is labeled with an acceptor fluorophore, TR-FRET can occur in the presence of the calprotectin antigen (analyte) (FIG. 2). The increase in FRET signal of the acceptor is proportional to the level of calprotectin present in the patient’s sample (e.g., stool or fecal sample) as interpolated from a known amount of calprotectin calibrators (FIG. 3).

### Example 2: Illustrates Preparing a Predetermined Standardized Curve

The disclosure provides a method for preparing and storing a predetermined standardized curve for an assay. A plurality of lyophilized calibrator compositions comprising a known amount of analyte (e.g., calprotectin) together with for example, an anti-calprotection antibody (MAB-1) labeled with a donor fluorophore and a second antibody (MAB-2) labeled with acceptor fluorophore are lyophilized. The plurality of lyophilized calibrators is place in an assay device, which is a multiwall plate. The lyophilized product is reconstituted with buffer. The assay device is incubated in an analyzer. Next, the method includes obtaining an optical signal for each of the plurality of standard calibrator compositions to generate a plurality of optical signals; and thereafter preparing a standardized curve of the analyte from the plurality of optical signals. An increase in FRET signal of the acceptor is proportional to the level of calprotectin present in the sample (e.g., stool or fecal sample) as for example, shown FIG. 3. The calibration curve is stored or encoded in a tag or barcode and associated with the lyophilized product.

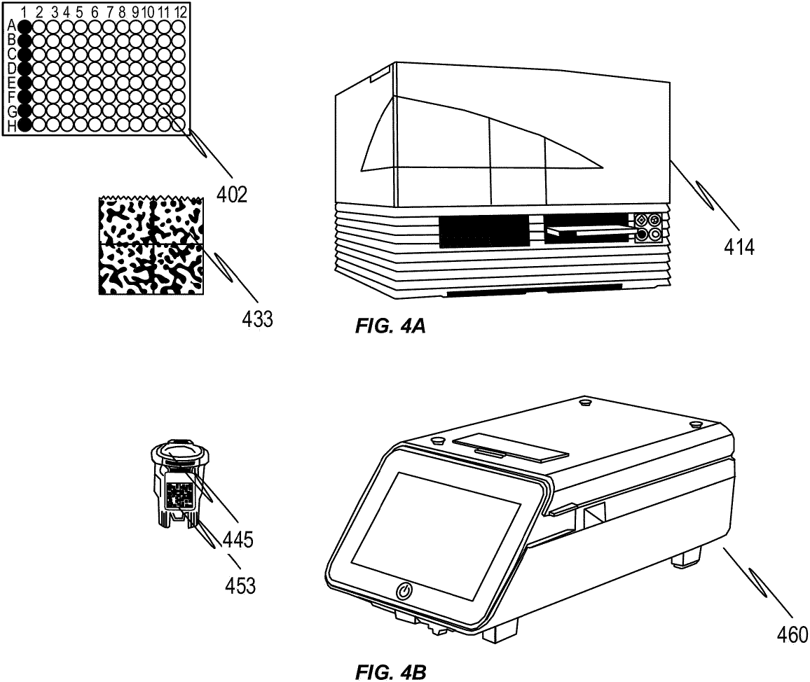

FIG. 4A shows an assay device 402 with a plurality of lyophilized calibrators (9 calibrators, labeled A-H). The analyzer 414 can produce an optical signal for each of the plurality of standard calibrator compositions to generate a plurality of optical signals; and thereafter a standardized curve of the analyte from the plurality of optical signals is calculated. The standardized curve is stored on a label such as a barcode 433.

The disclosure further provides a method for adjusting the predetermined standardized curve of an analyte for an assay by measuring a signal for a calibrator composition, wherein the calibrator composition comprises a known amount of the analyte within the predetermined standardized curve. The user can obtain a ratio of the signal for the calibrator composition, (actual readout divided by predetermined readout which is expected). The ratio is used to adjust the predetermined standardized curve.

As shown in FIG. 4B, the user can measure a signal on an instrument 460 for a calibrator 445. The calibrator signal should fall on the predetermined calibration curve stored on the barcode 453. Any change in the predicted concentration value versus actual concentration value is a ratio as described. Small off-sets or adjustments can be made to unknown concertation of analyte based on the ratio. In other words, an unknown analyte concentration is determined according to the adjusted predetermined standardized curve based on ratio.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for preparing and storing or assigning a predetermined standardized curve for an assay, the method comprising:
(a) preparing a plurality of lyophilized calibrator compositions in an assay device, wherein the assay device has a plurality of wells or is a single cuvette;
(b) incubating the assay device in an analyzer;
(c) obtaining an optical signal for each of the plurality of standard calibrator compositions to generate a plurality of optical signals;
(d) preparing a standardized curve of the analyte from the plurality of optical signals; and
(e) storing or assigning the predetermined standardized curve into a label or barcode for the assay device.

2. The method of claim 1, wherein each calibrator composition contains a first antibody having a first epitope for an analyte; a second antibody having a second epitope for the analyte, and a known amount of analyte.

3. The method of claim 2, wherein a fluorophore acceptor is attached to the first antibody and a fluorophore donor is attached to the second antibody.

4. The method of claim 1, wherein each calibrator composition contains an antibody for the analyte and a known amount of analyte.

5. The method of claim 4, wherein a fluorophore acceptor is attached to the antibody and a fluorophore donor is attached to the analyte.

6. The method of claim 1, wherein the assay device is a member selected from the group consisting of a 96, 384 and 1536 well plate.

7. The method of claim 1, wherein the assay device is a cuvette.

8. The method of claim 1, wherein the analyte is a member selected from the group consisting of a protein, an autoantibody, a nucleic acid and a vitamin.

9. A method for adjusting a predetermined standardized curve of an analyte for an assay, the method comprising:
(a) measuring a signal for a calibrator composition, wherein the calibrator composition comprises a known amount of the analyte within the predetermined standardized curve;
(b) obtaining a ratio of the signal for the calibrator composition; and
(c) adjusting the predetermined standardized curve according to the ratio obtained.

10. The method of claim 9, further comprising (d) optionally determining an unknown analyte concentration according to the adjusted predetermined standardized curve.

11. The method of claim 9, wherein the calibrator composition is a plurality of calibrator compositions.

12. The method of claim 9, wherein the predetermined output can be loaded onto a barcode, RFID tag or label.

13. The method of claim 9, wherein the ratio is used to normalize different analyzers.

14. A lyophilized product, the lyophilized product containing:
a fluorophore acceptor attached to a first antibody having a first epitope for an analyte;
a fluorophore donor attached to a second antibody having a second epitope for the analyte; and
a known amount of analyte.

15. The lyophilized product of claim 1, wherein the lyophilized product is a bead.

16. The lyophilized product of claim 15, wherein the bead is a plurality of beads, wherein each bead has a different concentration of analyte.

17. The lyophilized product of claim 1, wherein the analyte is a member selected from the group consisting of a protein, a nucleic acid, an autoantibody and a vitamin.

18. The lyophilized product of claim 17, wherein the analyte is a protein.

19. The lyophilized product of claim 17, wherein the analyte is an autoantibody.

20. The lyophilized product of claim 17, wherein the analyte is a vitamin.
